(19) United States
(12) Patent Application Publication
(10) Pub. No.: US 2024/0164424 A1
(43) Pub. Date: May 23, 2024

(54) Hericium erinaceus DESIGNATED AS STRAIN M2-102-10 AND COMPOSITIONS, PRODUCTS, AND METHODS THEREOF

(71) Applicant: M2 Ingredients, Inc., Vista, CA (US)

(72) Inventors: Samuel Andrasko, Lakeside, CA (US); Julie Daoust, Vista, CA (US); Steven Lee Farrar, Vista, CA (US)

(21) Appl. No.: 18/514,846

(22) Filed: Nov. 20, 2023

Related U.S. Application Data
(60) Provisional application No. 63/427,548, filed on Nov. 23, 2022.

(51) Int. Cl. A23L 31/00; A23L 2/38; A23P 10/22; C12N 1/14

(52) U.S. Cl. CPC A23L 31/00; A23L 2/38; A23P 10/22; C12N 1/145

(57) ABSTRACT

Embodiments of the present disclosure include Hericium erinaceus fungus designated as strain M2-102-10 as deposited with the ATCC Patent Depository under the Budapest Treaty (10801 University Blvd, Manassas, VA 20110), on Nov. 7, 2023, with the unofficial ATCC Patent Deposit Designation No. PTA-127680 and the official deposit date of ______ and the official Patent Deposit Designation No. ______. In some embodiments, the improved Hericium erinaceus fungus designated as strain M2-102-10 is characterized by having optimized concentrations of bioactive compounds. In some embodiments, a Beta-glucan concentration is 20 percent or greater.

### CROSS REFERENCE TO RELATED APPLICATIONS

This patent application claims the priority benefit of U.S. Provisional Patent Application Ser. No. 63/427,548, filed on Nov. 23, 2022, and titled “Hericium erinaceus DESIGNATED AS STRAIN M2-102-10”. The aforementioned disclosure is hereby incorporated by reference in its entirety including all patent deposit material(s) and all references cited therein.

### FIELD OF THE TECHNOLOGY

The present invention belongs to the field of microbial technology. More specifically. More specifically, embodiments of the disclosure relate to an improved strain of Hericium erinaceus (also called lion's mane mushroom, mountain-priest mushroom or bearded tooth fungus).

### BRIEF SUMMARY

Embodiments of the present disclosure include a Hericium erinaceus fungus designated as strain M2-102-10 as deposited with the ATCC Patent Depository under the Budapest Treaty (10801 University Blvd, Manassas, VA 20110), on Nov. 7, 2023, with the unofficial ATCC Patent Deposit Designation No. PTA-127680 and the official deposit date of and the official Patent Deposit Designation No.

Some embodiments include a Hericium erinaceus fungus designated as strain M2-102-10, characterized by a phenotype having a solid mass of fruitbodies on top of a substrate.

Some embodiments include a Hericium erinaceus fungus designated as strain M2-102-10, characterized by having fruiting initiation between sixteen to nineteen days after inoculation.

Some embodiments include a Hericium erinaceus fungus designated as strain M2-102-10, characterized by being optimized to grow on oats.

Some embodiments include a Hericium erinaceus fungus designated as strain M2-102-10, characterized having a beta-glucan content of 20% to 40%.

Some embodiments include a Hericium erinaceus fungus designated as strain M2-102-10, characterized by having a beta-glucan content of at least 20%.

Some embodiments include a Hericium erinaceus fungus designated as strain M2-102-10, characterized by having a beta-glucan content of 40%.

Some embodiments include a Hericium erinaceus fungus designated as strain M2-102-10, characterized by having a beta-glucan content of 20% to 35%.

Some embodiments include a Hericium erinaceus fungus designated as strain M2-102-10, characterized by having a beta-glucan content of 25% to 35%.

Some embodiments include a Hericium erinaceus fungus designated as strain M2-102-10, characterized by having a beta-glucan content of 20% to 30%.

Some embodiments include a Hericium erinaceus fungus designated as strain M2-102-10, characterized by having a beta-glucan content of 25% to 30%.

Some embodiments include a Hericium erinaceus fungus designated as strain M2-102-10, characterized by having a beta-glucan content of 20% to 25%.

Some embodiments include a Hericium erinaceus fungus designated as strain M2-102-10, characterized by having a beta-glucan content of 25%.

Some embodiments include a Hericium erinaceus fungus designated as strain M2-102-10, including an optimized concentration of at least one of hericenone A, hericenone B, hericenone E, hericenone F, hericenone J, isohericerin, hericerin, and N-De phenylethyl isohericerin.

Some embodiments include a Hericium erinaceus fungus designated as strain M2-102-10, including an optimized concentration of erinacine C.

Some embodiments include a Hericium erinaceus fungus designated as strain M2-102-10, including an optimized concentration of erinacine D.

Some embodiments include a Hericium erinaceus fungus designated as strain M2-102-10, including an optimized concentration of erinacine E.

Some embodiments include a Hericium erinaceus fungus designated as strain M2-102-10, including an optimized concentration of erinacine C, erinacine D, and erinacine E.

Some embodiments include a Hericium erinaceus fungus designated as strain M2-102-10, including an optimized concentration of hericenone E.

Some embodiments include a Hericium erinaceus fungus designated as strain M2-102-10, including an optimized concentration of hericenone A, hericenone B, hericenone E, and hericenone F.

Some embodiments include a Hericium erinaceus fungus designated as strain M2-102-10, including an optimized concentration of isohericerin.

Some embodiments include a Hericium erinaceus fungus designated as strain M2-102-10, including an optimized concentration of hericerin.

Some embodiments include a Hericium erinaceus fungus designated as strain M2-102-10, including an optimized concentration of N-De phenylethyl isohericerin.

Some embodiments include a method for propagating a Hericium erinaceus fungus designated as strain M2-102-10 using liquid spawn, the method including: culturing a mother culture; culturing a production culture, the production culture being a sub-culture of the mother culture; creating a liquid master inoculum using the production culture; diluting the liquid master inoculum to create a diluted liquid master inoculum; creating a biomass product using the diluted liquid master inoculum; slicing the biomass product for dehydration to create a sliced biomass product; dehydrating the sliced biomass product to create a dehydrated sliced biomass product; milling the dehydrated sliced biomass product to select a designated particle size of the dehydrated sliced biomass product; and screening the designated particle size of the dehydrated sliced biomass product.

Some embodiments include a method for propagating a Hericium erinaceus fungus designated as strain M2-102-10 for using grain spawn, the method including: culturing a mother culture; culturing a production culture, the production culture being a sub-culture of the mother culture; creating a master spawn using the production culture; creating a production spawn using the master spawn; creating a biomass product using the production spawn; slicing the biomass product for dehydration to create a sliced biomass product; dehydrating the sliced biomass product to create a dehydrated sliced biomass product; milling the dehydrated sliced biomass product to select a designated particle size of the dehydrated sliced biomass product; and screening the designated particle size of the dehydrated sliced biomass product.

Some embodiments include a method for propagating a Hericium erinaceus fungus designated as strain M2-102-10, further including an edible product, the edible product including the designated particle size of the dehydrated sliced biomass product.

Some embodiments include a product including Hericium erinaceus fungus designated as strain M2-102-10 including at least one of mycelium, spawn, inoculum, fresh mushrooms, processed mushrooms, mushroom extracts and fractions, mushroom pieces, and colonized substrates including grain, and friable particulate matter.

Some embodiments include a Hericium erinaceus fungus designated as strain M2-102-10, in a powder form.

Some embodiments include a Hericium erinaceus fungus designated as strain M2-102-10, in a capsule form.

Some embodiments include a Hericium erinaceus fungus designated as strain M2-102-10, in a hot beverage form.

Some embodiments include a Hericium erinacens fungus designated as strain M2-102-10, characterized by having no bitterness in taste.

### BRIEF DESCRIPTION OF THE FIGURES

The patent or application file contains at least one photograph executed in color. Copies of this patent or patent application publication with color photograph(s) will be provided by the Office upon request and payment of the necessary fee.

FIG. 1 photograph showing morphological characteristics of Hericium erinaceus fungus designated as strain M2-102-10, according to some embodiments of the present disclosure.

FIG. 2 is another photograph showing the morphological characteristics of Hericium erinaceus fungus designated as strain M2-102-10, according to some embodiments of the present disclosure.

FIG. 3 is another photograph showing the morphological characteristics of Hericium erinaceus fungus designated as strain M2-102-10, according to some embodiments of the present disclosure.

FIG. 4 is a photomicrograph of hyphal aggregation of Hericium erinaceus fungus designated as strain M2-102-10, according to some embodiments of the present disclosure.

FIG. 5 is photograph showing bioreactor bags containing Hericium erinaceus fungus designated as strain M2-102-10, according to some embodiments of the present disclosure.

FIG. 6 is photograph showing fruiting bodies of Hericium erinaceus fungus designated as strain M2-102-10 in a bioreactor bag at three times magnification, according to some embodiments of the present disclosure.

FIG. 7 is photograph showing fruiting bodies of Hericium erinaceus fungus designated as strain M2-102-10 in a bioreactor bag at five times magnification, according to some embodiments of the present disclosure.

FIG. 8A is a flowchart illustrating a method for propagating a Hericium erinaceus fungus designated as strain M2-102-10 using liquid spawn, according to some embodiments of the present disclosure.

FIG. 8B is a flowchart illustrating a method for propagating a Hericium erinaceus fungus designated as strain M2-102-10 using grain spawn, according to some embodiments of the present disclosure.

FIG. 9 displays a table of some of the biologically active metabolites of a Hericium erinaceus fungus designated as strain M2-102-10, according to some embodiments of the present disclosure.

FIG. 10 a laboratory report of results for Beta-glucan testing of samples of Hericium erinaceus fungus designated as strain M2-102-10, according to some embodiments of the present disclosure.

FIG. 11 another laboratory report of results for Beta-glucan testing of samples of Hericium erinaceus fungus designated as strain M2-102-10, according to some embodiments of the present disclosure.

### DETAILED DESCRIPTION

Exemplary embodiments of the present technology include a Hericium erinaceus fungus designated as strain M2-102-10 as deposited with the ATCC Patent Depository under the Budapest Treaty (10801 University Blvd, Manassas, VA 20110), on Nov. 7, 2023, with the unofficial ATCC Patent Deposit Designation No. PTA-127680.

In various embodiments, Strain M2-102-10 (also known as HE-120) is grown for use in Organic Mushroom (“Om’) products (Lion's Mane Capsules, Lion's Mane Powder, Master Blend Powder, Master Blend Capsules, Brain Fuel plus Mushroom, Mushroom Hot Chocolate, Mushroom Coffee, Mushroom Coffee Latte) and may be sold as a bulk powder under M2 Ingredients, Inc. For example, this species is used in products with cognitive benefits.

Hericium erinaceus (Lion's Mane mushroom) is grown through the full life cycle of the mushroom and includes all of the bioactive compounds produced during the mycelial stage including erinacines as well as during the fruiting body stage including hericenones. Both of these compounds erinacines and hericenones have been researched for their cognitive health benefits. Lion's Mane is also highly regarded for its immune support and gastro-protective functionalities.

Hericium erinaceus (Lion's Mane mushroom) has active compounds that affect the synthesis and activity of neurotrophic factors that are important for the brain and nervous system. Neurotrophic factors have potent biological activities, such as preventing neuronal death and promoting neurite outgrowth that is essential for maintenance and organization of neurons. For example, a nootropic is defined as a substance that enhances cognition and memory and facilitates learning. Several studies have reported important nootropic bioactivities in Lion's Mane including the induction of Nerve Growth Factor (NGF) bio-synthesis, inhibition of the cytotoxicity of the beta-amyloid peptide and protection against neuronal cell death by oxidative stress. Functional deficiency of NGF is related to memory loss and plays a role in the etiology of Alzheimer's. Research on the low-molecular weight compounds found in Lion's Mane that promote NGF such as hericenones, amycenone and erinacines have reported that the molecules are small enough to cross the blood-brain barrier in order to effect increased bio-synthesis of NGF.

Beta-glucans are polymers of ß-D-glucose. They constitute part of cell walls of bacteria and plants, mainly algae and cereals, as well as microscopic fungi including Hericium erinaceus. Beta-glucans have been studied for their various health benefits, particularly in relation to immune system support and cardiovascular health. Some potential health benefits associated with beta-glucans include immune system support, cardiovascular health, blood sugar control, and weight management, just to name a few.

In various embodiments of the present technology, phenotypical characteristics of the Hericium erinaceus fungus designated as strain M2-102-10 include the following. Embodiments produce a solid mass of fruitbody on the top of grain substrate from 1 to 2.7 pounds. In some embodiments, fruits best at 62 to 65 degrees Fahrenheit with nearly no detectable bitterness in taste whereas other strains evaluated and reports from wild strains are bitter to the taste. Beta-glucan content range of 20-35% depending on a number of days of culture before harvest. The specification for this ingredient is set to a minimum of 20% beta glucans to have erinacine C, erinacine D, and erinacine E at various concentration ranges. The Hericium erinaceus fungus designated as strain M2-102-10 of claim 1, comprising an optimized concentration of isohericerin, hericerin and N-De phenylethyl isohericerin in various embodiments.

In various embodiments Beta-glucan concentration is 20% or greater and this specific point of at least 20% is critical or special to the operability of Strain M2-102-10. Strain M2-102-10 with a Beta-glucan concentration of 20% or greater produces a new and unexpected result, which is different in kind and not merely in degree.

In some embodiments a grain spawn with Strain M2-102-10 (also known as HE-120) is specifically used for production. For example, a grain spawn of made from sterilized grains that have been inoculated with a live mycelium culture of Strain M2-102-10. These inoculated grains are consumed by the growing mass of mycelium of Strain M2-102-10. In various embodiments, a liquid spawn of strain M2-102-10 is used as an alternative method.

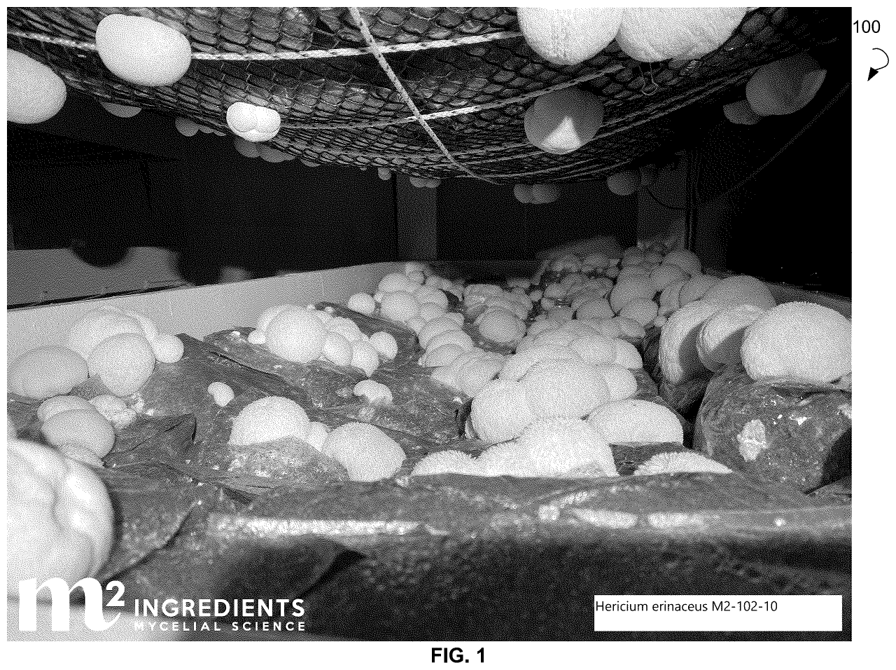

FIG. 1 photograph 100 showing morphological characteristics of Hericium erinaceus fungus designated as strain M2-102-10, according to some embodiments of the present disclosure. For example, the Hericium erinaceus fungus designated as strain M2-102—may be characterized by a phenotype having a solid mass of fruitbodies on top of a substrate. Some embodiment may be characterized by having fruiting initiation between sixteen to nineteen days after inoculation, and in various embodiments may be characterized by being optimized to grow on oats.

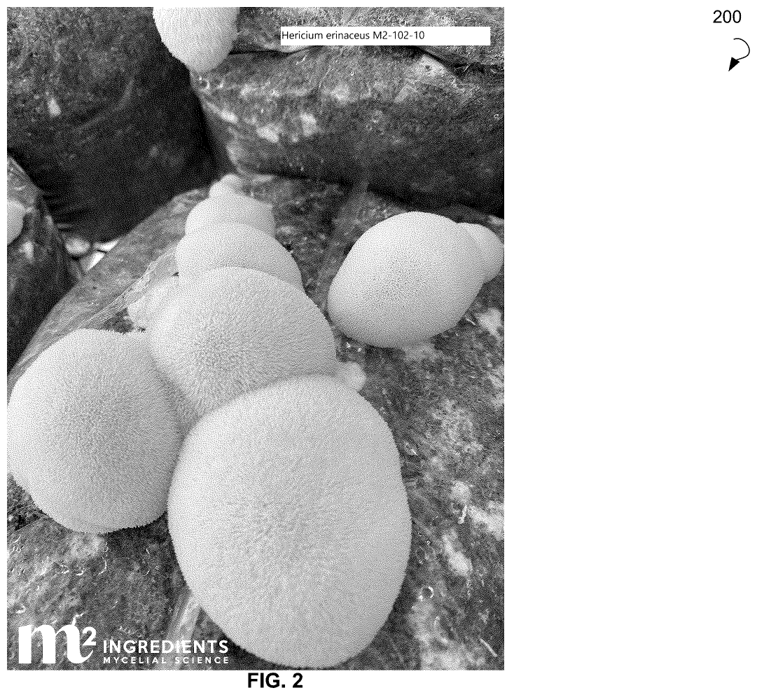

FIG. 2 is another photograph 200 showing the morphological characteristics of Hericium erinaceus fungus designated as strain M2-102-10, according to some embodiments of the present disclosure. For example, the Hericium erinaceus fungus designated as strain M2-102—may be characterized by a phenotype having a solid mass of fruitbodies on top of a substrate. Some embodiment may be characterized by having fruiting initiation between sixteen to nineteen days after inoculation, and in various embodiments may be characterized by being optimized to grow on oats.

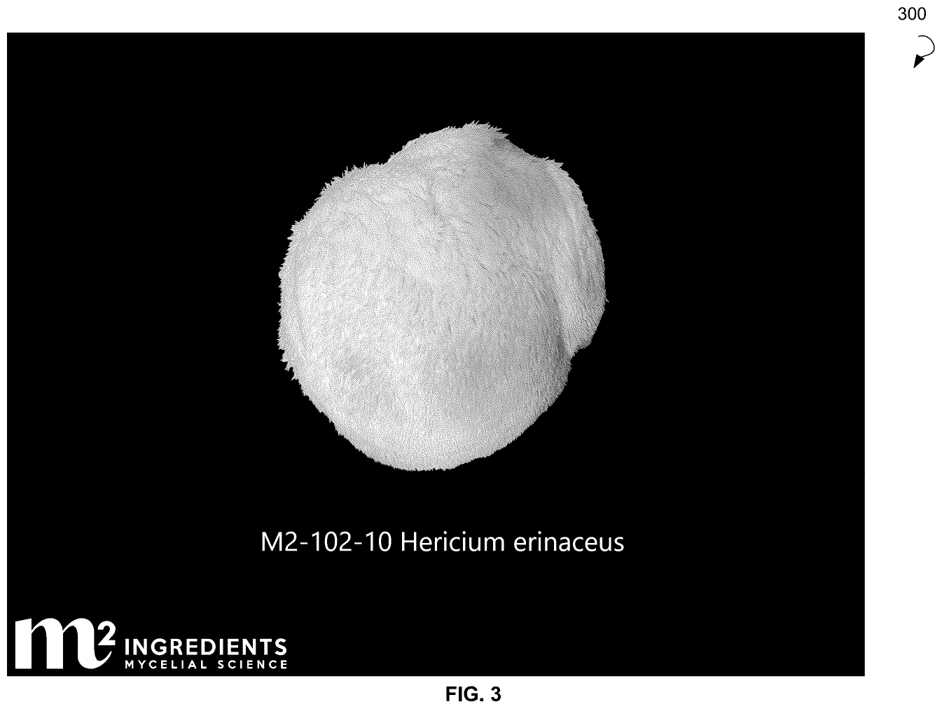

FIG. 3 is another photograph 300 showing the morphological characteristics of Hericium erinaceus fungus designated as strain M2-102-10, according to some embodiments of the present disclosure.

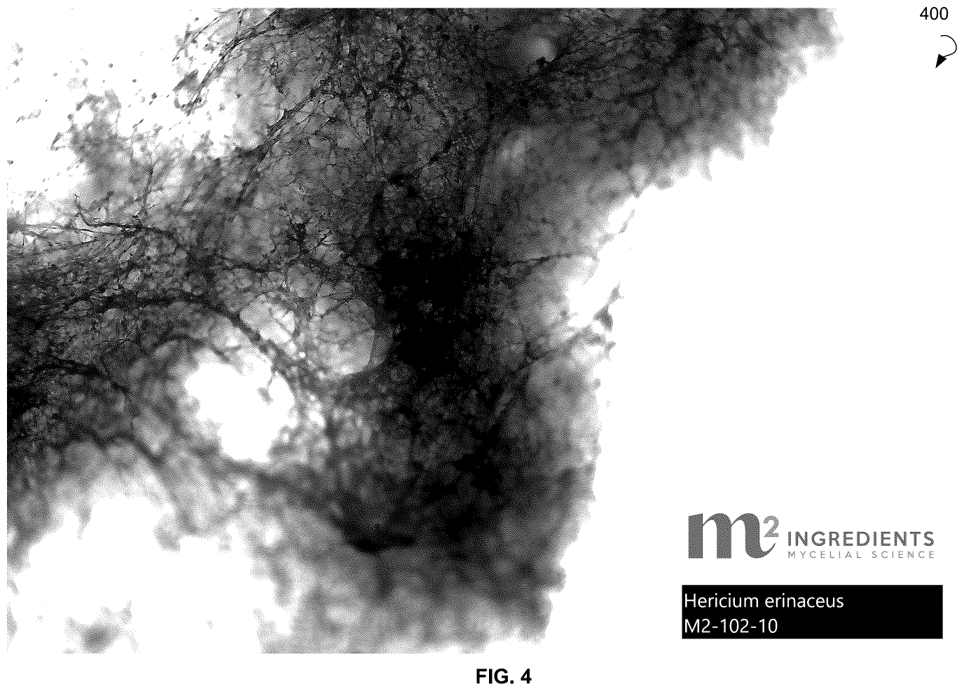

FIG. 4 is a photomicrograph 400 of hyphal aggregation of Hericium erinaceus fungus designated as strain M2-102-10, according to some embodiments of the present disclosure. For example, hyphal aggregation includes the initial fruitbody development.

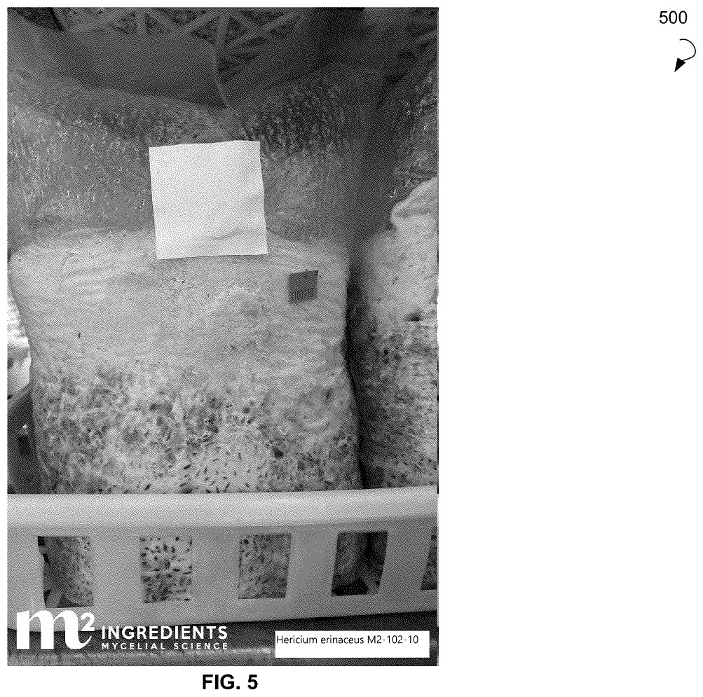

FIG. 5 is photograph 500 showing bioreactor bags containing Hericium erinaceus fungus designated as strain M2-102-10, according to some embodiments of the present disclosure. For example, bioreactor bags containing Hericium erinaceus fungus designated as strain M2-102-10 may be characterized by a phenotype having a solid mass of fruitbodies on top of a substrate. Some embodiment may be characterized by having fruiting initiation between sixteen to nineteen days after inoculation, and in various embodiments may be characterized by being optimized to grow on oats.

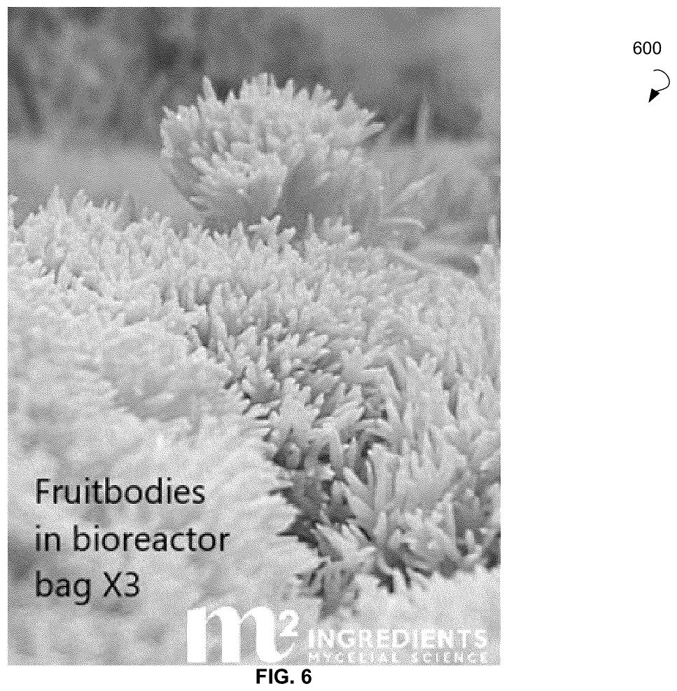

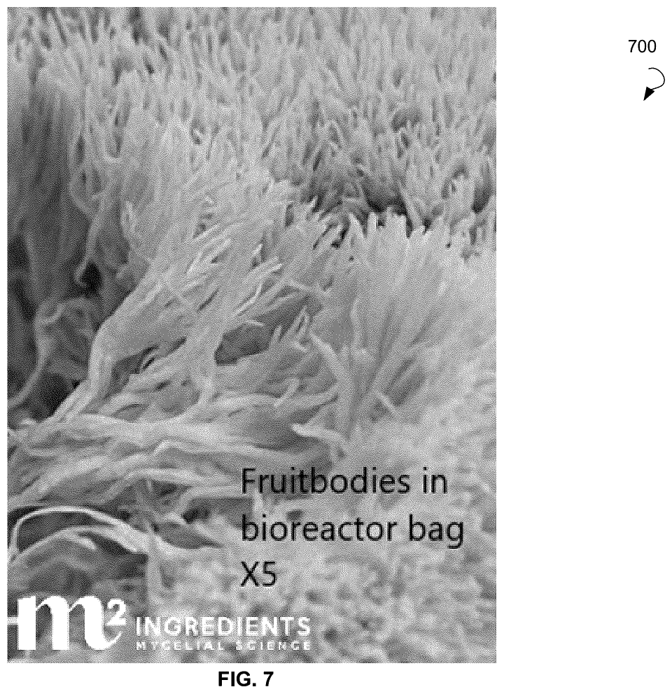

FIG. 6 is photograph 600 showing fruiting bodies of Hericium erinaceus fungus designated as strain M2-102-10 in a bioreactor bag at three times magnification, according to some embodiments of the present disclosure. FIG. 7 is photograph 700 showing fruiting bodies of Hericium erinaceus fungus designated as strain M2-102-10 in a bioreactor bag at five times magnification, according to some embodiments of the present disclosure.

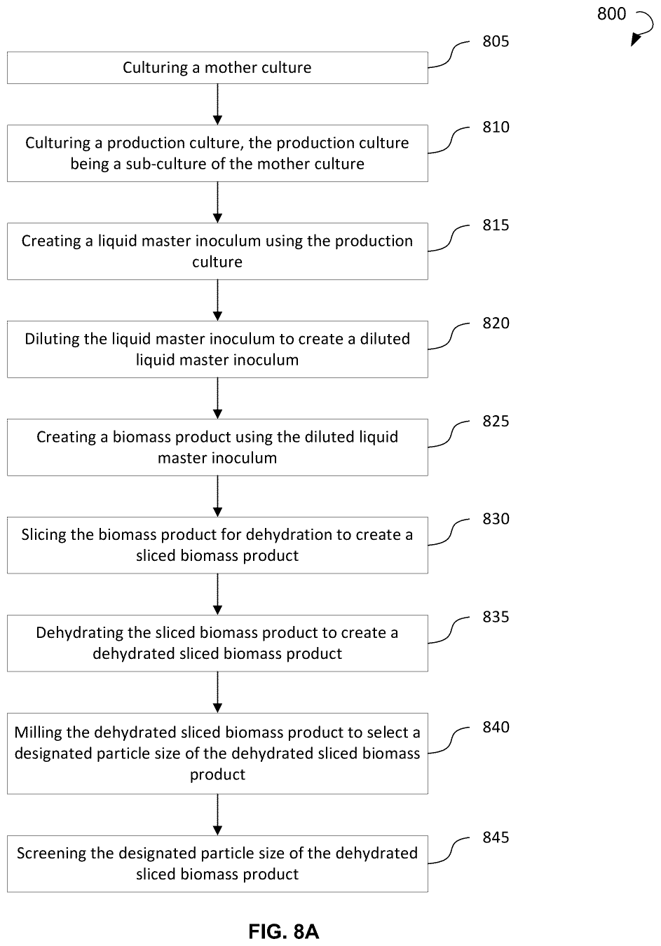

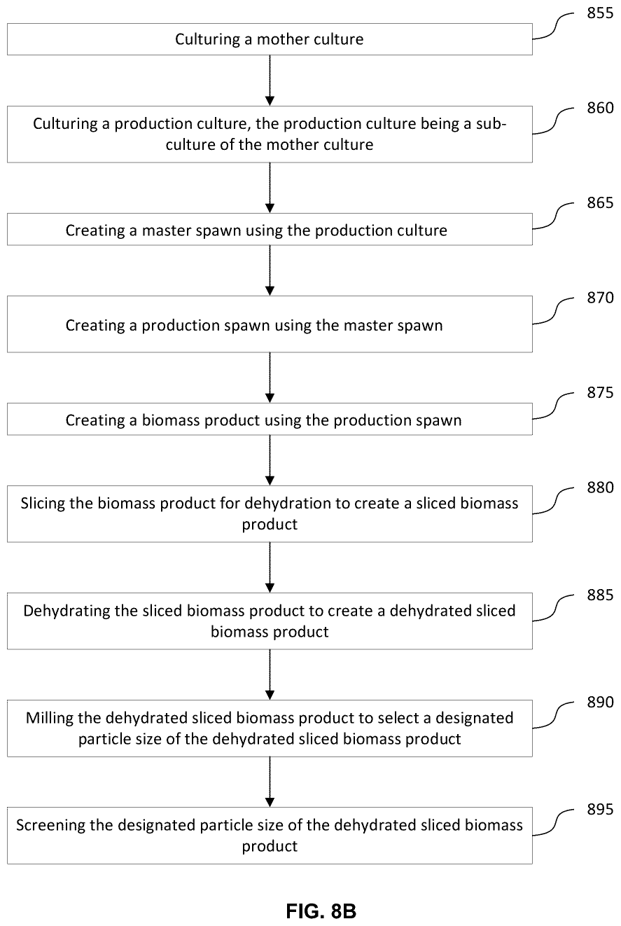

FIG. 8A is a flowchart that describes a method for propagating a Hericium erinaceus mycelium using liquid spawn, according to some embodiments of the present disclosure. In some embodiments, at step 805, the method may include culturing a mother culture. At step 810, the method may include culturing a production culture, the production culture being a sub-culture of the mother culture. At step 815, the method may include creating a liquid master inoculum using the production culture. At step 820, the method may include diluting the liquid master inoculum to create a diluted liquid master inoculum. In some embodiments, at step 825, the method may include creating a biomass product using the diluted liquid master inoculum. At step 830, the method may include slicing the biomass product for dehydration to create a sliced biomass product. At step 835, the method may include dehydrating the sliced biomass product to create a dehydrated sliced biomass product. At step 840, the method may include milling the dehydrated sliced biomass product to select a designated particle size of the dehydrated sliced biomass product. At step 845, the method may include screening the designated particle size of the dehydrated sliced biomass product. More details according to various embodiments are disclosed below.

FIG. 8B is a flowchart illustrating a method for propagating a Hericium erinaceus fungus designated as strain M2-102-10 using grain spawn, according to some embodiments of the present disclosure. FIG. 8B is a flowchart of an example method for propagating a Hericium erinaceus fungus designated as strain M2-102-10 using grain spawn, the method comprising the following steps. At step 855, culturing a mother culture. At step 860, culturing a production culture, the production culture being a sub-culture of the mother culture. At step 865, creating a master spawn using the production culture. At step 870, creating a production spawn using the master spawn. At step 875, creating a biomass product using the production spawn. At step 880, slicing the biomass product for dehydration to create a sliced biomass product. At step 885, dehydrating the sliced biomass product to create a dehydrated sliced biomass product. At step 890, milling the dehydrated sliced biomass product to select a designated particle size of the dehydrated sliced biomass product. At step 895, screening the designated particle size of the dehydrated sliced biomass product. More details according to various embodiments are disclosed below. In some embodiments, FIG. 8B is a preferred embodiment for propagating the Hericium erinaceus fungus designated as strain M2-102-10 using grain spawn because of high quality product being produced using grain spawn. Additional, details according to various embodiments are disclosed below.

FIG. 8A is a flowchart that describes a method for propagating a Hericium erinaceus mycelium using liquid spawn, according to some embodiments of the present disclosure. In some embodiments, at step 805, the method may include culturing a mother culture. For example, culturing of mother cultures (i.e., preserved stock cultures) may be “the first step in the process”. Fresh cultures of the Hericium erinaceus mycelium of the M2-102-10 strain are cultured on potato dextrose agar and grown for a period of 9 to 16 days until a culture diameter of 30 to 40 mm is obtained. For example, 5 mm×5 mm square sections of the healthiest part of the mother culture is cut from the leading edge of growth of the hyphae and placed into a 1.5 ml to 2.0 ml cryogenic storage vial of 10 to 20 percent glycerol solution and cryogenically stored under Liquid Nitrogen (LN2). At the same time, 2 mm square sections of the same mother culture material is transferred to 10 to 20 culture tubes equal or greater than 25 mm×150 mm in size filled with a nutrient agar containing a carbon/nitrogen/vitamin/mineral mix and subsequently stored under refrigeration between 34 to 40 degrees Fahrenheit. Cultures tubes are sub-cultured every 6 to 12 months to new culture tubes to verify that the cultures are still active and healthy and to preserve the phenotypic qualities of the original cultures. The cryogenically stored cultures previously discussed above may be recovered and sub-cultured into new culture tubes for storage. Furthermore, any culture with one or more observed unwanted phenotypical variations are not used. Additionally, only growth representative of the mother culture is used for inoculum.

In some embodiments at step 810, the method may include culturing a production culture, the production culture being a sub-culture of the mother culture. For example, culturing of production cultures may be “the second step in the process”. To start the production process, for example, mother cultures are pulled from refrigeration and sub-cultured onto 4 nutrient agar plates. Production cultures are allowed to grow for 9 to 16 days until a growth diameter of 30 to 40 mm is obtained. The healthiest and most representative tissue of a production culture is sub-cultured in step 815.

In some embodiments at step 815, the method may include creating a liquid master inoculum using the production culture. For example, creation of a master spawn also known as “First (1^{st}) generation grain spawn” (G1's) may be “the third step in the process”. An exemplary protocol is as follows. For example, one pound (lb.) of hydrated hulled oats are hydrated to a 50 to 60% moisture content in static and/or dynamic water tanks at 190 to 205 degrees Fahrenheit for 30 min to 60 min, and filled into an appropriate clear autoclavable bioreactor bag. For example, the bioreactor bag comprising a polypropylene, or high density polyethylene, or a blend of polypropylene/high density polyethylene, which is fitted with a microporous filter patch or strip material for gas exchange. The oats are sterilized in an autoclave for an appropriate time and temperature to fully render any present microorganisms inactive. The bioreactor bags are cooled in a High Efficiency Particulate Air (HEPA) filtrated and refrigerated in a cooldown room until the grain is equal to or greater than 90 degrees Fahrenheit. Once cooled, the bioreactor bags are delivered to a HEPA filtrated laminar flow hood and a laboratory technician subcultures a 10 mm by 10 mm section of the healthiest tissue from step 810 to each individual master spawn bag. Each master spawn bag is allowed to incubate at 67 to 70 degrees Fahrenheit for 12 to 18 days. During incubation each master spawn bag is shaken once a 3 cm colony from around the inoculation site occurs. The master spawn bag is left to incubate until the grain is fully consolidated with the mycelium and then the shaken again. The master spawn bag is then placed under refrigeration at 34 to 40 degrees Fahrenheit to slow down the metabolism of the culture and stall the growth. The master spawn is then monitored for contamination and regrowth every 3 days until the master spawn is sent to create production spawn in step 820.

In some embodiments at step 820, the method may include diluting the liquid master inoculum to create a diluted liquid master inoculum. For example, creation of production spawn “second generation spawn” (G2's) may be “the 4th step in the process”. An exemplary protocol is as follows. For example, 7 lbs. to 8.5 lbs. of hydrated hulled oats, hydrated to a 50 to 60% moisture content in static and/or dynamic water tanks at 190 to 205 Fahrenheit for 30 min to 60 min is filled into an appropriate clear autoclavable bioreactor bag comprised of a polypropylene or high density polyethylene or a blend of Polypropylene/High density polyethylene that is fitted with a microporous filter patch or strip material for gas exchange. The oats are subjected to sterilization in an autoclave for an appropriate time and temperature to fully render any present microorganisms inactive. The bags are cooled in a HEPA filtrated refrigerated cooldown room until the grain is equal to or less than 90 degrees Fahrenheit. Once cooled, the bags are delivered to a HEPA filtrated laminar flow hood and a laboratory technician subcultures the master grain bags to the production spawn at ratio of 1:10 to 1:20. The master grain bags are then incubated for 12 to 18 days.

In some embodiments, at step 825, the method may include creating a biomass product using the diluted liquid master inoculum. For example, creation of biomass product may be “the 5th step of the process”. An exemplary protocol is as follows. For example, 600 to 800 of 8.5 lbs. of hydrated hulled oats, hydrated to a 50-60% moisture content in static and/or dynamic water tanks at 190 to 205 Fahrenheit for 30 min to 60 min is filled into an appropriate clear autoclavable bioreactor bag comprised of a polypropylene or high-density polyethylene or a blend of Polypropylene/High density polyethylene that is fitted with a microporous filter patch or strip material for gas exchange. The oats are subjected to sterilization in an autoclave for an appropriate time and temperature to fully render any present microorganisms inactive. The bags are cooled in a HEPA filtrated refrigerated cooldown room until the grain is equal to or less than 90 degrees Fahrenheit. Once cooled the bags are delivered to a HEPA filtrated laminar flow hood and a laboratory technician subcultures the production spawn to the biomass bags to the biomass bags at ratio of 1:180 to 1:200. The bags are then incubated for 28 to 45 days. The bags contain the fruitbodies, mycelium and digested oats.

In some embodiments at step 830, the method may include slicing the biomass product for dehydration to create a sliced biomass product. For example, slicing “may be the 6th step of the process”. For instance, upon harvesting the biomass; the biomass is sliced though a dicer into smaller pieces and placed upon dehydration trays for dehydration.

In some embodiments at step 835, the method may include dehydrating the sliced biomass product to create a dehydrated sliced biomass product. For example, dehydration of biomass product may be “the 7th step of the process”. For instance, the trays of biomass product is loaded into a static dehydrator and dried for approximately 24 hours and/or into a dynamic continuous dehydrator for approximately 30 to 60 minutes to a moisture content of less than 5%. This process is carefully watched to prevent altering of the taste of the final product.

In some embodiments at step 840, the method may include milling the dehydrated sliced biomass product to select a designated particle size of the dehydrated sliced biomass product. For example, milling may be “the 8th step of the process”. For instance, the dried biomass is put though a mill and screened to a designated particle size.

In some embodiments at step 845, the method may include screening the designated particle size of the dehydrated sliced biomass product. For example, the dried biomass is screened to a designated particle size and bulk packed for customers.

Is some embodiments, FIG. 8B is a preferred embodiment for propagating the Hericium erinaceus fungus designated as strain M2-102-10 using grain spawn because of high quality product being produced using grain spawn. Using grain spawn of FIG. 8B includes step 865, creating a master spawn using the production culture (1st generation spawn). Furthermore, at step 865, creating a production spawn using the master spawn (2nd generation spawn. Moreover, at step 875, creating a biomass product using the production spawn (3rd generation biomass product).

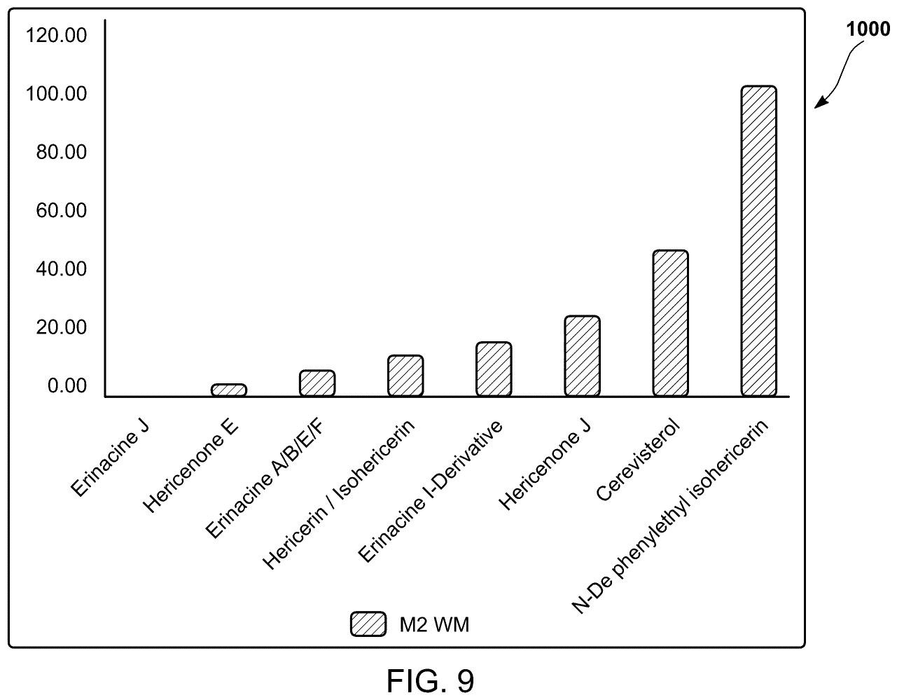

FIG. 9 displays a table of some of the biologically active metabolites of a Hericium erinaceus fungus designated as strain M2-102-10, according to some embodiments of the present disclosure. The data was generated using the SCIEX 5600 Quadrupole TOF mass spec for a full molecular analysis. The System User Guide is incorporated by reference, which is titled, “TripleTOF 5600/5600+ System, System User Guide”, RUO-IDV-05-7040-E, March 2022 (147 pages). AB Sciex Pte. Ltd, Blk33, #4-6 Marsiling Industrial Estate Road 3, Woodlands Central Industrial Estate, Singapore 739256. The X axis of the table shows biologically active metabolites of a Hericium erinaceus fungus designated as strain M2-102-10. The Y axis of the table shows relative intensity of biologically active metabolites of the Hericium erinaceus fungus designated as strain M2-102-10 compared to a control sample.

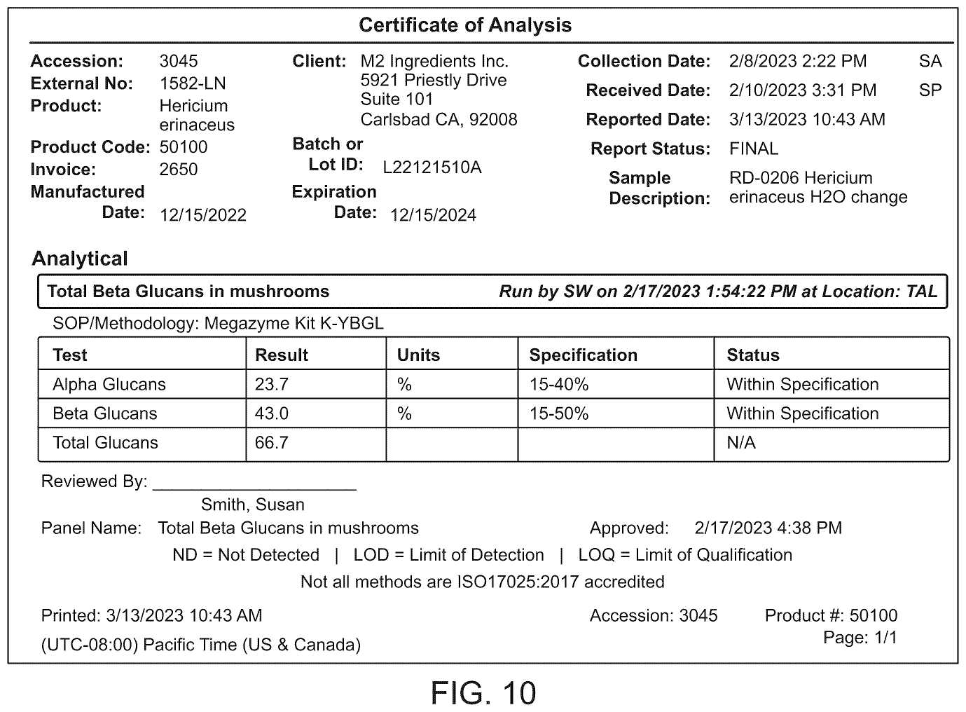

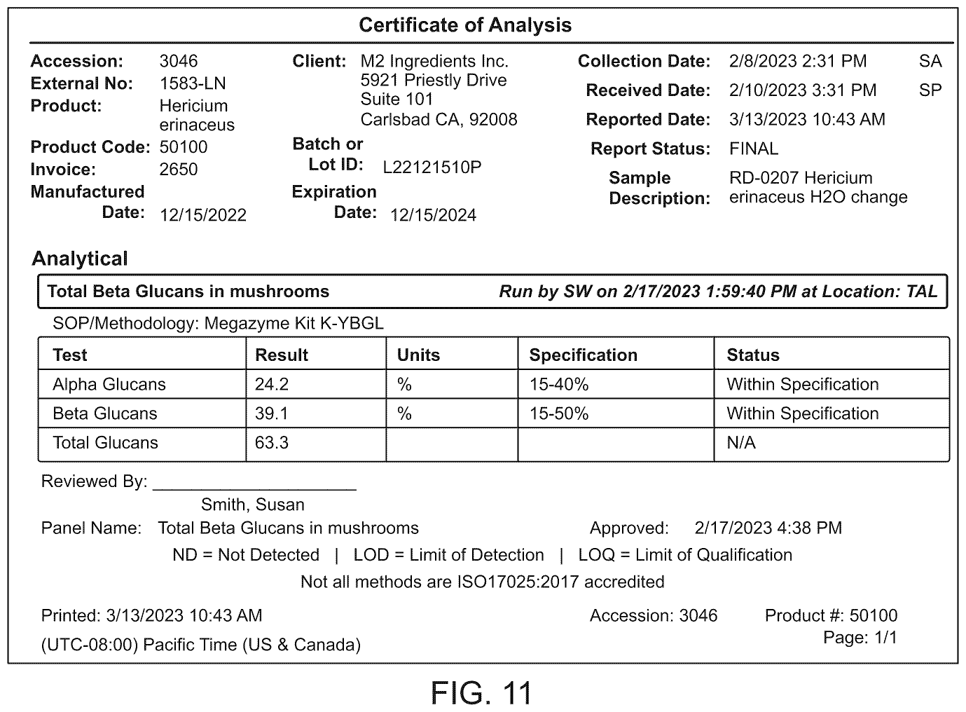

FIG. 10 a laboratory report of results for Beta-glucan testing of samples of Hericium erinaceus fungus designated as strain M2-102-10, according to some embodiments of the present disclosure. FIG. 11 another laboratory report of results for Beta-glucan testing of samples of Hericium erinaceus fungus designated as strain M2-102-10, according to some embodiments of the present disclosure.

### Example—Detection of Biologically Active Metabolites

Samples of Hericium erinaceus fungus designated as strain M2-102-10 were obtained in solid form for data analysis of select biologically active metabolites. The samples were removed from room temperature storage and mixed thoroughly. Sample processing proceeded with 0.1 g of respective samples were weighed in triplicates on a calibrated analytical balance, 1.2 ml of IPA:MEOH:H20 (40:40:20) was added to the samples for “Sample set A samples,” and 1.2 ml of EtOH:H20 (1:1) was added to the samples for “Sample set B samples.” After mixing gently, the samples were sonicated for 50 min. Sample processing proceeded with dilution 5: 100 μl of the sample was mixed with 400 μl of Acetonitrile to get a 5× dilution, vortex mix for 15 minutes at 25 degrees Celsius, centrifuge for 20 minutes, 190 μL of dilution, 100 sample plus 10 μl of IS. The samples were then proceeded to be analyzed with Liquid chromatography-mass spectrometry (LC-MS) analysis using the SCIEX 5600 Quadrupole TOF mass spec for a full molecular analysis as described herein. The results are shown in FIG. 9 that displays a table of some of the biologically active metabolites.

### Example—Detection of Beta-Glucan

Samples of Hericium erinaceus fungus designated as strain M2-102-10 were obtained in solid form for data analysis detection of Beta-glucans. Samples were processed using SOP Megazyme Kit K-YBGL. Megazyme's Beta-Glucan Assay Kit (Yeast & Mushroom), (K-YBGL), is a colorimetric method used for the measurement and analysis of 1,3:1,6-Beta-D-glucan (plus 1,3-Beta glucan, if present) and 1,4:1,6-Alpha-glucan (starch/glycogen) in yeast and mushrooms. The β-GLUCAN (Yeast and Mushroom) ASSAY PROTOCOL K-YBGL is incorporated by reference, which is titled, “8-GLUCAN (Yeast and Mushroom) ASSAY PROTOCOL”, 2023, Neogen Corporation; August 2023, Megazyme. (11 pages). This method is measures Yeast Beta-Glucan in g/100 g on an “as is” basis. Exemplary results are shown in FIG. 9 and FIG. 10 as obtained from Twin Arbor Analytical Laboratory 3474 Empresa Dr. Suite 140, San Luis Obispo, CA 93401.

While various embodiments have been described above, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the technology as defined by the appended claims. Thus, the breadth and scope of a preferred embodiment should not be limited by any of the above-described exemplary embodiments.

What is claimed is:

1. A Hericium erinaceus fungus designated as strain M2-102-10 with an unofficial ATCC Patent Deposit Designation No. PTA-127680 and an official Patent Deposit Designation No. ______.

2. The Hericium erinaceus fungus designated as strain M2-102-10 of claim 1, characterized by a phenotype having a solid mass of fruitbodies on top of a substrate.

3. The Hericium erinaceus fungus designated as strain M2-102-10 of claim 1, characterized by having fruiting initiation between sixteen to nineteen days after inoculation.

4. The Hericium erinaceus fungus designated as strain M2-102-10 of claim 1, characterized by being optimized to grow on oats.

5. The Hericium erinaceus fungus designated as strain M2-102-10 of claim 1, characterized having a beta-glucan content of 20% to 40%.

6. The Hericium erinaceus fungus designated as strain M2-102-10 of claim 1, characterized by having a beta-glucan content of at least 20%.

7. The Hericium erinaceus fungus designated as strain M2-102-10 of claim 1, characterized by having a beta-glucan content of 40%.

8. The Hericium erinaceus fungus designated as strain M2-102-10 of claim 1, characterized by having a beta-glucan content of 20% to 35%.

9. The Hericium erinaceus fungus designated as strain M2-102-10 of claim 1, characterized by having a beta-glucan content of 25% to 35%.

10. The Hericium erinaceus fungus designated as strain M2-102-10 of claim 1, characterized by having a beta-glucan content of 20% to 30%.

11. The Hericium erinaceus fungus designated as strain M2-102-10 of claim 1, characterized by having a beta-glucan content of 25% to 30%.

12. The Hericium erinaceus fungus designated as strain M2-102-10 of claim 1, characterized by having a beta-glucan content of 20% to 25%.

13. The Hericium erinaceus fungus designated as strain M2-102-10 of claim 1, characterized by having a beta-glucan content of 25%.

14. The Hericium erinaceus fungus designated as strain M2-102-10 of claim 1, comprising an optimized concentration of at least one of hericenone A, hericenone B, hericenone E, hericenone F, hericenone J, isohericerin, hericerin, and N-De phenylethyl isohericerin.

15. The Hericium erinaceus fungus designated as strain M2-102-10 of claim 1, comprising an optimized concentration of erinacine C.

16. The Hericium erinaceus fungus designated as strain M2-102-10 of claim 1, comprising an optimized concentration of erinacine D.

17. The Hericium erinaceus fungus designated as strain M2-102-10 of claim 1, comprising an optimized concentration of erinacine E.

18. The Hericium erinaceus fungus designated as strain M2-102-10 of claim 1, comprising an optimized concentration of erinacine C, erinacine D, and erinacine E.

19. The Hericium erinaceus fungus designated as strain M2-102-10 of claim 1, comprising an optimized concentration of hericenone E.

20. The Hericium erinaceus fungus designated as strain M2-102-10 of claim 1, comprising an optimized concentration of hericenone A, hericenone B, hericenone E, and hericenone F.

21. The Hericium erinaceus fungus designated as strain M2-102-10 of claim 1, comprising an optimized concentration of isohericerin.

22. The Hericium erinaceus fungus designated as strain M2-102-10 of claim 1, comprising an optimized concentration of hericerin.

23. The Hericium erinaceus fungus designated as strain M2-102-10 of claim 1, comprising an optimized concentration of N-De phenylethyl isohericerin.

24. A method for propagating a Hericium erinaceus fungus designated as strain M2-102-10 using liquid spawn, the method comprising:
culturing a mother culture;
culturing a production culture, the production culture being a sub-culture of the mother culture;
creating a liquid master inoculum using the production culture;
diluting the liquid master inoculum to create a diluted liquid master inoculum;
creating a biomass product using the diluted liquid master inoculum;
slicing the biomass product for dehydration to create a sliced biomass product;
dehydrating the sliced biomass product to create a dehydrated sliced biomass product;
milling the dehydrated sliced biomass product to select a designated particle size of the dehydrated sliced biomass product; and
screening the designated particle size of the dehydrated sliced biomass product.

25. A method for propagating a Hericium erinaceus fungus designated as strain M2-102-10 for using grain spawn, the method comprising:
culturing a mother culture;
culturing a production culture, the production culture being a sub-culture of the mother culture;
creating a master spawn using the production culture;
creating a production spawn using the master spawn;
creating a biomass product using the production spawn;
slicing the biomass product for dehydration to create a sliced biomass product;
dehydrating the sliced biomass product to create a dehydrated sliced biomass product;
milling the dehydrated sliced biomass product to select a designated particle size of the dehydrated sliced biomass product; and
screening the designated particle size of the dehydrated sliced biomass product.

26. The method for propagating a Hericium erinaceus fungus designated as strain M2-102-10 of claim 25, further comprising an edible product, the edible product comprising the designated particle size of the dehydrated sliced biomass product.

27. A product comprising Hericium erinaceus fungus designated as strain M2-102-10 comprising at least one of mycelium, spawn, inoculum, fresh mushrooms, processed mushrooms, mushroom extracts and fractions, mushroom pieces, and colonized substrates including grain, and friable particulate matter.

28. The Hericium erinaceus fungus designated as strain M2-102-10 of claim 27, in a powder form.

29. The Hericium erinaceus fungus designated as strain M2-102-10 of claim 27, in a capsule form.

30. The Hericium erinaceus fungus designated as strain M2-102-10 of claim 27, in a hot beverage form.

31. The Hericium erinaceus fungus designated as strain M2-102-10 of claim 27, characterized by having no bitterness in taste.
